(19) United States
(12) Patent Application Publication
(10) Pub. No.: US 2019/0307903 A1
(43) Pub. Date: Oct. 10, 2019

(54) METHOD FOR MANUFACTURING A BIOCOMPATIBLE FLUID COMPRISING A POWDER OF MAGNETIC PARTICLES, BIOCOMPATIBLE FLUID COMPRISING A POWDER OF MAGNETIC PARTICLES

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE (CNRS), Paris Cedex (FR)

(72) Inventors: Robert MOREL, GRENOBLE (FR); Bernard DIENY, GRENOBLE (FR); Hélène JOISTEN, GRENOBLE (FR)

(21) Appl. No.: 16/376,297

(22) Filed: Apr. 5, 2019

(30) Foreign Application Priority Data
Apr. 5, 2018 (FR) 1852971

(51) Int. Cl. A61K 49/00; A61K 33/26; B22F 1/02; A61K 9/50; A61K 49/10

(52) U.S. Cl. CPC A61K 49/0002; A61K 33/26; A61K 49/0093; A61K 9/5094; A61K 49/101; B22F 1/02

(57) ABSTRACT

A method for manufacturing a biocompatible fluid including a powder of magnetic particles of elongated shape having a magnetic shape anisotropy and having a final granulometry, the final granulometry being defined by a first average size of the particles in a first direction and a second average size in a second direction different from the first direction, the final granulometry further being defined by a first distribution width of the first sizes and a second distribution width of the second sizes, the method including from a powder of magnetic particles having an initial granulometry different from the final granulometry, modification of the initial granulometry by milling and/or by sintering of the powder until the final granulometry is obtained; introduction of the powder of magnetic particles into a biocompatible fluid.

### CROSS REFERENCE TO RELATED APPLICATIONS

This application claims priority to French Patent Application No. 1852971, filed Apr. 5, 2018, the entire content of which is incorporated herein by reference in its entirety.

### FIELD

The invention belongs to the field of biocompatible fluids including a powder of magnetic particles for the application of a low frequency magnetic-mechanical vibration. The invention firstly relates to a manufacturing method including a powder of magnetic particles having a given granulometry. The invention secondly relates to a biocompatible fluid including a powder of magnetic particles for the application of a low frequency magnetic-mechanical torque. The biocompatible fluid according to the invention may be used for the destruction of cancerous cells by the low frequency vibration of particles near to the targeted cells.

### BACKGROUND

In the biotechnologies field, superparamagnetic magnetic particles of very small size (at the most several tens of nanometres) are increasingly used in diverse applications such as targeted drug delivery, the separation of molecules or cells in suspension, treatments of cancer by hyperthermia, cellular tissue engineering or as contrast agents, see for example the document “Applications of magnetic nanoparticles in biomedicine”, published in J. Phys. D: Appl. Phys. 36, R167, 2003 of Q. A. Pankhurst et al.

The particles the most often used are small superparamagnetic particles covered with an envelope, inorganic such as for example silica, or polymer such as for example dextran or polyethylene glycol (PEG), ensuring the protection and the biocompatibility thereof. The particles may be functionalised by covering them with a layer of molecules, for example to attach themselves in a targeted manner onto a cell or a tissue, to transport an active compound or a drug, or to ensure the dispersion or the mobility thereof. Depending on the targeted applications, these molecules may ensure the dispersion or the mobility thereof. Depending on the targeted applications, these particles may have dimensions of several nanometres (MRI contrast agent), or of several micrometres when they are composed of an assembly of magnetic particles, embedded in a matrix.

New therapeutic approaches, notably for the treatment of cancer, are based on exercising mechanical actions on biological species thanks to magnetic particles which may be internalised, in contact, or near to the media to treat (see for example the document “Biofunctionalized magnetic-vortex microdiscs for targeted cancer-cell destruction” published in Nature Materials 9, 165 (2010), of D. Kim et al.). The mechanical force exerted by a magnetic particle is the consequence of the magnetic torque that is exerted thereon, which for its part results from the application of a field that is variable in direction or in modulus (see the document “Actuating Soft Matter with Magnetic Torque”, published in Adv. Funct. Mater. 26, 3859 (2016), of R. M. Erb et al.).

It is known to use low frequency magnetic-mechanical vibration of magnetic particles for therapeutic applications. The particles used for these applications have a size close to a micrometre and do not include metal oxides. The magnetic fields used to make the particles vibrate oscillate at a frequency that is at the most several tens of Hz.

Magnetic particles of metal oxides of small size—typically several tens of nm—are used for other applications such as hyperthermia. In these applications, magnetic fields of several hundreds of kHz are used to heat the particles.

The magnetic particles required for applications that involve inducing magnetic-mechanical vibrations for the destruction of cells must have a certain number of essential characteristics:
They must have low magnetisation at zero field, to avoid agglomeration;
They must be able to be actuated by a field of low amplitude;
For particles where shape anisotropy dominates, they must be of anisotropic shape so that the magnetic torque induces a rotation of the particle (and not uniquely a rotation of its magnetisation);
They must have a size of the order of a micrometre so that the rotation of the particle induces a noticeable mechanical effect on the targeted cell or medium;
They must be biocompatible.

Several documents of the prior art describe magnetic particles for the application of a low frequency vibration, but the known particles do not comprise all of the ideally required characteristics.

The patent application WO 2005011810, “Magnetic particles for therapeutic treatment”, filed by Oxford Instruments Superconductivity Limited, describes a method for destroying cells by mechanical rupture, where localised particles are subjected to low intensity magnetic fields and of which the orientation varies at low frequency. The particles described by this document are preferentially particles of low magnetic anisotropy, of which the largest dimension is less than 0.5 pm. The shape of these particles is preferably prolate ellipsoidal, or oblate ellipsoidal. The magnetisation of the particles is stabilised by the intrinsic magnetocrystalline anisotropy and/or by the shape anisotropy. The size and shape parameters are chosen to minimise the magnetic field necessary for the application of a force of 100 pN, considered necessary to bring about a notable effect on the cells. No method for manufacturing these particles is disclosed by this document.

The patent application WO 2006134365, “Method of providing magnetised particles at a location” filed by Oxford Instruments Molecular Biotools, describes magnetic particles with vortex structure, optimised for the application of a force. The particles are composed of Supermalloy (Ni78Fe18Mo4), Permalloy (Ni80Fe20) or nickel. The particles described by this document may have different shapes:
Spherical particles of diameter comprised between 50 nm and 200 nm;
Quasi-spherical particles;
Particles having a large dimension and a small dimension, the large dimension being comprised between 50 nm and 200 nm, the small dimension being above 5 nm and less than the large dimension.

Thanks to their vortex structure, these particles have very low magnetisation at zero field, which favours good dispersion, while limiting aggregation due to magnetic interaction. In addition, these particles have high magnetic susceptibility, which reduces the magnetic field required to make them move. However these particles are not biocompatible because they all contain nickel.

Other documents of the prior art consider the use of iron oxide particles for destroying cells by mechanical action, but in regimes of high frequency or magnetic field intensity or instead by the use of strong magnetic field gradients.

The patent application WO 200137721 describes a method for inducing cell death under the effect of particles of less than 100 nm. The particles are in contact with the cell or internalised by endocytosis, following the application of a static magnetic field. The action mechanism of the particles is not explained, but the use of a static magnetic field excludes the vibration of the particles.

The patent application WO 2014038829 “Method for selectively activating magnetic nanoparticle and selectively activated magnetic nanoparticle” filed by the University of Seoul belongs to the field of magnetic hyperthermia. This document describes a selective mode of activating and heating magnetic particles including:

a) the application of a first non-oscillating or DC magnetic field to define the resonance frequency of the particles,

b) the application of a second high frequency magnetic field applied at a given angle with respect to the DC field in such a way as to induce a precession of the magnetisation which constitutes the activation of the particles, which, in this context corresponds to a heating effect. The frequency of the magnetic field bringing about the activation is of the order of 1 MHz. The magnetic particles have a vortex structure, being able to be constituted of maghemite (γ-Fe₂O₃), magnetite (Fe₃O₄), barium ferrite (BaFeO) or CoFe₂O₄. The particles have a diameter between 40 nm and 200 nm.

The patent application WO 2001017611, “Device for therapeutic purposes on human tissue, for influencing injected magnetic particles with an alternating electro-magnetic gradient field” also belongs to the hyperthermia field. This document describes a device generating an alternating high frequency field (up to 30 MHz) to increase the heating and/or kinetic effect of the particles. The particles used are constituted of iron oxide and have a diameter comprised between 0.1 nm and 300 nm.

It is also possible to distinguish magnetic particles known in the prior art according to the technique used for the manufacture thereof. “Top-down” and “bottom-up” techniques may then be distinguished.

FIG. 1 schematically illustrates a “top-down” method (see for example the documents “Self-polarization phenomenon and control of dispersion of synthetic antiferromagnetic nanoparticles for biological applications”, published in Applied Physics Letters 97, 253112 (2010) of H. Joisten et al. and the document “Ferromagnetic microdisks as carriers for biomedical applications”, published in Journal of Applied Physics 105, 07B306 (2009) of E. A. Rozhkova et al.). The main steps consist in depositing layers of resins D, defining the particles by lithography L, developing and opening the resin and depositing the films or the magnetic multilayers G, and releasing the particles by lift-off LO. Several alternatives are possible but all proceed from such top-down approaches. The particles thereby obtained are flat, often circular (in disc shape), with a large dimension of the order of a micron and of thickness of the order of 100 nm.

On account of the techniques implemented, the manufacture of the particles is extremely expensive. Moreover, this technique has a low production efficiency compared to needs: of the order of several 100 μg per substrate (wafer) are obtained, whereas several grams of particles are necessary to treat a tumour of several cm³. This low efficiency is inherent to the process of manufacturing on the surface of a substrate. The low quantity produced is however sufficient for in vitro tests, enabling the initial optimisation of the particles.

On account of the lithography, these particles have a very small dispersion in shape and in size. This characteristic is interesting for applications of contrast agent for magnetic imaging type but is not required to exert forces or torques on biological species, in particular cells, which contributes to the fact that this elaboration method is unsuited compared to the needs.

A strong constraint for these particles is the necessity of ensuring their dispersion in a liquid. In practice, a necessary condition is that the magnetisation in the absence of applied field is low. For micronic particles, this is obtained when the magnetic structure is such that the internal magnetisation, at zero field, is compensated over the whole of the particle. This may be achieved in several ways: either by using particles having a so-called micromagnetic flux closure configuration, or by using structures in disordered domains leading to zero magnetisation such as for example in polycrystalline magnetite discs.

Concerning particles with flux closure, this magnetic flux closure may be obtained for example in discs of Ni80Fe20 alloy, of micronic size and of thickness of the order of one hundred or so nanometres. Examples of particles with flux closure are illustrated in FIG. 2. In these structures, the magnetic configuration in zero field is a vortex contained in the plane of the particle, with for sole remanent magnetisation the low magnetisation of the vortex core, see for example the document “Biofunctionalized magnetic-vortex microdiscs for targeted cancer-cell destruction” published in Nature Materials 9, 165 (2010), of D. Kim et al.

In practice, the obtaining of the structure vortex is limited to a reduced range of sizes and shapes, which requires for the production of the particles using the top-down elaboration means already mentioned. The susceptibility of these particles is sufficiently high so that under application of a moderate magnetic field (several tens of milliteslas), a net magnetisation appears, which can bring about a significant magnetic-mechanical torque.

Since the nickel contained in these vortex particles is not a biocompatible material, the particles are encapsulated between two films of gold during manufacture. The films of gold that cover the two faces of the vortex particles further enable functionalisation, notably by the use of thiols.

Another example of particles with magnetic flux closure is constituted of so-called synthetic antiferromagnetic multilayers, or synthetic antiferromagnetics (SAF), in which a magnetic material and a non-magnetic metallic material alternate ensuring antiferromagnetic coupling between adjacent magnetic layers. An example is constituted by CoFe/Ru multilayers. Examples of these particles are illustrated in FIG. 3 (see also the documents “Self-polarization phenomenon and control of dispersion of synthetic antiferromagnetic nanoparticles for biological applications”, published in Applied Physics Letters 97, 253112 (2010) of H. Joisten and the document “High-Moment Antiferromagnetic Nanoparticles with Tunable Magnetic Properties”, published in Advanced Materials 20, 1479 (2008) of W. Hu et al.).

The thickness of the non-magnetic metal (usually ruthenium) is chosen such that an antiferromagnetic coupling develops between the magnetic layers. This thickness is for this purpose typically chosen between 0.3 and 0.9 nm. In the absence of field, the magnetisations of the successive magnetic layers are antiparallel and the overall magnetisation is zero—this is then known as synthetic antiferromagnetic, SAF. These particles have the advantage of having a susceptibility greater than vortex particles hence, for a given magnetic field, a greater exerted magnetic torque (see the document “Comparison of dispersion and actuation properties of vortex and synthetic antiferromagnetic particles for biotechnological applications”, published in Applied Physics Letters 103, 132412 (2013) of S. Leulmi et al.).

Here again, since the ruthenium and cobalt or nickel contained in these SAF particles is not biocompatible, the particles are encapsulated between two films of gold during manufacture.

For circular discs (vortex or SAF) released from their substrate (that is to say for example in suspension in a liquid), the minimisation of the energy in a turning field leads to orienting gradually the plane of the particle parallel to the rotational plane of the field. The rotation of the magnetisation in the plane of the circular particle no longer exerts (in this case) but a very weak magnetic torque.

To circumvent this effect it is possible to used magnetic particles that are globally flat but of non-circular shape (for example elliptical) or even irregular, or particles having an anisotropy perpendicular to the plane of the layers such that the magnetisation of the particle points spontaneously out of the plane of the particle and not in the plane. Such particles with perpendicular magnetisation and synthetic antiferromagnetics are for example constituted of multilayers composed of Ta, Pt, CoFeB and Ru (see for example the document “Highly tunable perpendicularly magnetized synthetic antiferromagnets for biotechnology applications”, published in Applied Physics Letters 107, 012403 (2015), of T. Vemulkar et al.).

The particles with perpendicular magnetisation are manufactured using the top-down techniques described previously. Conversely, they have a much greater number of layers, which significantly lengthens the manufacturing time and are extremely sensitive to problems of roughness of the layers. Their manufacturing costs are even higher than that of vortex particles. The particles with perpendicular magnetisation are themselves also constituted of materials which, for certain, are not biocompatible.

None of the particles of the prior art comprises all the characteristics cited previously for the efficient destruction of cancerous cells by low frequency vibration.

Even if particles in vortex have given good results in terms of destruction of cancerous cells, they contain non-biocompatible materials, which makes their use very difficult or even impossible.

Generally speaking, the magnetic particles of the prior art are obtained by clean room elaboration methods, which are intrinsically slow, complex and costly.

Numerous types of magnetic particles are moreover manufactured by chemical synthesis. Conversely, the particles thereby produced are very small (up to 20 nm diameter), they are most generally spherical (sometimes cubic) and have structures that are either homogenous, or concentric, which do not make it possible to obtain the desired magnetic properties.

At present, magnetic particles are not available that are at one and the same time biocompatible, magnetically anisotropic, of micronic size, able to exert considerable torques on biological species and being able to be manufactured in large quantity at low cost.

### SUMMARY

To resolve at least partially the problems of the prior art, the present invention firstly relates to a method for manufacturing a biocompatible fluid including magnetic metal oxide particles obtained from powders. The metal oxide particles are biocompatible and may be produced in large quantity and at low cost.

An aspect of he invention thus firstly relates to a method for manufacturing a biocompatible fluid comprising a powder of magnetic particles of elongated shape having a magnetic shape anisotropy and having a final granulometry, the final granulometry being defined by a first average size of the particles in a first direction and a second average size in a second direction different from the first direction, the second average size being less than 1.5 times the first average size, the final granulometry further being defined by a first distribution width of the first sizes and a second distribution width of the second sizes, the method including the following steps:
From a powder of magnetic particles having an initial granulometry different from the final granulometry, modification of the initial granulometry by milling and/or by sintering of the powder until the final granulometry is obtained;
Introduction of the powder of magnetic particles into a biocompatible fluid;
   the first average size of the magnetic particles being comprised between 0.2 μm and 10 μm and the distribution width of the first sizes representing at least 30% of the value of the first average size.

According to an embodiment the first average size of the particles is beneficially comprised between 0.2 μm and 5 μm.

Granulometry of a powder is taken to mean the statistical distribution of the sizes of the particles forming the powder. For example, the granulometry of a powder may be characterised by the average size of its particles. Another parameter describing the granulometry of a powder is the width of the distribution of the sizes of the particles around the average size.

Magnetic particles are taken to mean grains of metal oxides, notably ferromagnetic iron oxides. Examples of such materials are magnetite Fe₃O₄ or maghemite γ-Fe₂O₃ or a mixture of these compounds.

Granulometry distributions may be characterised for example by scanning electron microscopy as illustrated in FIG. 5. This figure shows an assembly of grains of magnetite of irregular shape.

Size in a first direction is taken to mean size along the largest dimension of each of the particles. The average size in a first direction is then the average of these sizes along the largest dimension of each particle. Average size in a second direction different from the first is next taken to mean the average size of the particles in an arbitrary direction transversal to the first direction of each particle. These sizes may be obtained by analysis of scanning electron microscope images, as illustrated in FIG. 5. This figure shows an assembly of magnetite grains of irregular shape. An alternative technique consists in determining the sizes by dynamic light scattering (DLS).

Biocompatible fluid is taken to mean a fluid that does not interfere with or damage the biological medium in which it is used.

Beneficially, these materials are biocompatible and enable in vivo applications of the biocompatible fluid according to an embodiment of the invention.

Beneficially, these particles have low remanent magnetisation which ensures the dispersion thereof in liquid phase.

Beneficially, the particles have a magnetic shape anisotropy that comes from their high aspect ratio. This enables efficient vibration by magnetic-mechanical torque in the presence of a variable magnetic field.

The step of obtaining the final granulometry from an initial granulometry is carried out by milling or by sintering of the powder having the initial granulometry.

If the initial granulometry comprises an average size of the particles greater than the first average size of the final granulometry, the milling operation makes it possible to reduce the average size of the particles until the targeted granulometry is obtained. The milling operation may for example be carried out using a planetary ball mill.

Beneficially, in the case of a laboratory planetary ball mill, it is possible to manufacture in a single operation, which only lasts several hours, quantities of powder that range from less than one gram to several hundreds of grams.

These quantities are of several orders of magnitude greater than those obtained by top-down approaches and easily cover the needs for the treatment in parallel of several tumours or tissues, in animals or humans.

If the initial granulometry comprises an average size less than the first average size of the final granulometry, the initial average size of the particles may be increased by sintering. Sintering is taken to mean the process of partial or total melting of the particles due to the heating of the powder or to the application of a high pressure. The sintering operation is carried out at temperatures below the melting temperature of the material composing the magnetic particles.

Beneficially it is possible to apply to the starting powder having the initial granulometry a sequence of milling and sintering operations in order to obtain the final granulometry. In other words, the average size of the particles of the milled powder may be increased by sintering if it is too small. Vice-versa, the average size of the particles of the sintered powder may be reducing by milling if it is too large.

Beneificially, the method according to an embodiment of the invention makes it possible to produce quantities of particles such that it is possible to easily carry out in parallel physical-chemical characterisation studies such as size and surface potential measurements by dynamic light scattering (DLS).

The method according to an embodiment of the invention may fall within the scope of a more global process in which the initial powder could be derived from a more massive entity such as a pebble or strip.

The elongated shape of the particles and their magnetic shape anisotropy are such that, under application of a uniform and variable magnetic field, they undergo a magnetic torque which induces a force applied to the medium where the particle is located or on the substrate on which it rests or is fixed.

Beneficially, it is possible to use the force applied by the magnetic particles on the medium to destroy parts of the medium, such as cancerous cells.

Thanks to the fact that the distribution width of the first sizes is greater than or equal to 30% of the value of the first average size, the particles are not of homogeneous shapes and sizes. This makes it possible, during the placing in vibration with a magnetic field of given amplitude and frequency, to generate a whole range of torques and mechanical forces according to the size of each of the particles.

Beneficially, these particles are well suited to the application of forces or torques on biological species, especially when several milligrams, or even several grams of particles, are required.

The step of introducing the powder of magnetic particles into a biocompatible fluid is carried out by transfer of the magnetic particles from the milling or sintering vessel to a biocompatible fluid. As example of biocompatible fluids, physiological serum or PDMS may be cited. For injection into animals or humans, this fluid is for example a solution of PEG. For in vitro studies, the particles are added directly to the cell culture. An example of such a fluid is a solution of DMEM (Dulbecco Modified Eagle Medium) with GlutaMax and 10% of foetal calf serum (FCS).

The idea of an aspect of the invention results from the observation that magnetite and certain other iron oxides or compounds naturally have magnetic characteristics similar to those sought in vortex particles for biomedical applications, in a range of sizes and shapes much more extended than those known in the prior art. The manufacturing method according to an embodiment of the invention beneficially exploits the fact that a powder of iron compounds, providing that the average size of the particles is compatible with the application, is suited to triggering the death of cancerous cells by mechanical vibration, without a strict control of the shape homogeneity or that a precisely defined size are necessary. Compared to vortex particles manufactured by top-down method, the powders used in the method according to the invention are much easier and cheaper to produce and their implementation is facilitated by their biocompatibility.

The method according to one or more embodiments of the invention may also have one or more of the characteristics below, considered individually or according to all technically possible combinations thereof:
the first average size is comprised between 0.2 μm and 5 μm;
during the step of modification of the initial granulometry, the milling of the powder of magnetic particles having the initial granulometry is followed by the sintering of the powder resulting from the milling or the sintering of the powder of magnetic particles having the initial granulometry is followed by the milling of the powder resulting from the sintering;
the powder of final granulometry is of same chemical nature as the powder of initial granulometry;
the method according to an embodiment of the invention includes a step of chemical functionalisation of the particles;
the step of chemical functionalisation comprises an encapsulation of at least one part of the particles in an inorganic layer;
the inorganic layer is made of silica;
the step of chemical functionalisation includes a step of grafting of polymers on the surface of the particles or of the inorganic layer;
the grafted polymer includes polyethylene glycol (PEG);
the magnetic particles are grains including a metal oxide;
the method according to an embodiment of the invention further includes a step consisting in refining the size distribution of the particles in solution.

An aspect of the invention also relates to a biocompatible fluid comprising a powder of magnetic particles of elongated shape having a magnetic shape anisotropy and having a final granulometry, the final granulometry being defined by a first average size of the particles in a first direction and a second average size in a second direction different from the first direction, the second average size being less than 1.5 times the first average size, the final granulometry further being defined by a first distribution width of the first sizes and a second distribution width of the second sizes, the first average size of the magnetic particles being comprised between 0.2 pm and 10 μm and the distribution width of the first sizes representing at least 30% of the value of the first average size.

According to an embodiment, the first average size of the particles is beneficially comprised between 0.2 μm and 5 μm.

The magnitude of the magnetic-mechanical torque depends on the magnetic anisotropy. The particles must have a high magnetic anisotropy. Since this anisotropy often has for origin the shape anisotropy of the particle, a spherical particle would not be suited for the targeted applications.

If the particle is attached to a biological species, the torque that it undergoes induces, by lever effect, a mechanical force. The torque on a particle is proportional to the magnetic moment, thus to its volume: everything considered a larger particle is desirable. The mechanical force resulting from the torque also depends on other factors: 1) on the one hand, on the lever arm along which the torque is exerted, thus the particle shape; 2) in the event where the particle is anchored to the biological species, the transmitted mechanical force also depends on the bonding force. This anchoring may be obtained by the functionalisation of the particle with a suitable ligand.

The particles that make it possible to exert these mechanical actions must be sufficiently large to perturb the targeted biological species. The magnetic particles used in the biocompatible fluid according to the invention are of the order of a micron and make it possible to act on cells of which the size is of the order to ten or so microns.

The magnetic particles used in the biocompatible fluid according to an embodiment the invention, have several benefits:
Thanks to their low residual magnetisation the particles do not agglomerate in an irreversible manner when they are in solution, see for example “Self-polarization phenomenon and control of dispersion of synthetic antiferromagnetic nanoparticles for biological applications”, published in Applied Physics Letters 97, 253112 (2010) of H. Joisten et al.;
The particles are constituted of biocompatible magnetic materials so as not to induce a toxic reaction other than that induced by the desired magnetic-mechanical effects in the targeted organism;
The particles may be functionalised, for example to manage the mobility thereof, to ensure the stability in the medium where they are inserted, to prevent the agglomeration thereof and/or to enable a targeted action, notably therapeutic.

What is more, the exact shape of the particle, as well as the regularity of sizes and shapes in an embodiment of an embodiment of the invention, are not parameters requiring precise control. The magnetic-mechanical torque and the resulting forces more generally depend on the aspect ratio and on the overall dimensions. In most magnetic particles known in the prior art, the optimisation of the magnetic properties requires a rigorous control of the size and of the shape of the particles. For example within the context of contrast agents for magnetic resonance imaging, it is wished that the non-homogeneities observed in the spin relaxation times of the protons are caused by the variations in density of the surrounding tissues and the least possible by the distributions of properties of the contrast agents themselves. Beneficially, the present invention relaxes this constraint and allows the use of a set of particles of dispersed shapes and sizes, without prejudicing the attainment of the desired therapeutic objective.

The biocompatible fluid according to an embodiment of the invention may also have one or more of the characteristics below, considered individually or according to all technically possible combinations thereof:
the first average size of the particles is comprised between 0.2 μm and 5 μm;
the magnetic particles are grains including a metal oxide;
the metal oxide is a ferromagnetic iron oxide selected from a group including: magnetite, maghemite or a combination of these materials;
the magnetic particles are chemically functionalised;
the chemical functionalisation comprises the encapsulation of least one part of the magnetic particles in an inorganic layer;
the inorganic layer is made of silica;
the chemical functionalisation includes a step of grafting of polymers on the surface of the particles or of the inorganic layer;
the grafted polymer includes polyethylene glycol (PEG);
the particles are grains including a metal oxide.

### BRIEF DESCRIPTION OF THE DRAWINGS

Other characteristics of the invention will become clear from the description that is given thereof below, for indicative purposes and in no way limiting, while referring to the figures, among which:

FIG. 1 schematically represents the “top-down” type approach for manufacturing magnetic particles according to the prior art;

FIG. 2 represents scanning electron microscope images of magnetic particles produced using the technique illustrated in FIG. 1;

FIG. 3 illustrates examples of SAF type magnetic particles according to the prior art;

FIG. 4 schematically illustrates the steps of the method for manufacturing a biocompatible fluid according to an embodiment of the invention;

FIG. 5 is an electron microscope image of the particles of the biocompatible fluid according to the invention;

FIG. 6 represents a ball mill used to modify the granulometry of a powder;

FIG. 7 schematically illustrates the functionalisation of magnetite particles with organosilanes;

FIG. 8 schematically illustrates the functionalisation of magnetic particles with biotin/streptavidin-PE fluorophores;

FIG. 9 shows on the left particles according to an embodiment of the invention functionalised with a fluorophore and on the right non-functionalised particles according to the invention;

FIG. 10 illustrates the uniform magnetic field at the centre of a Halbach cylinder.

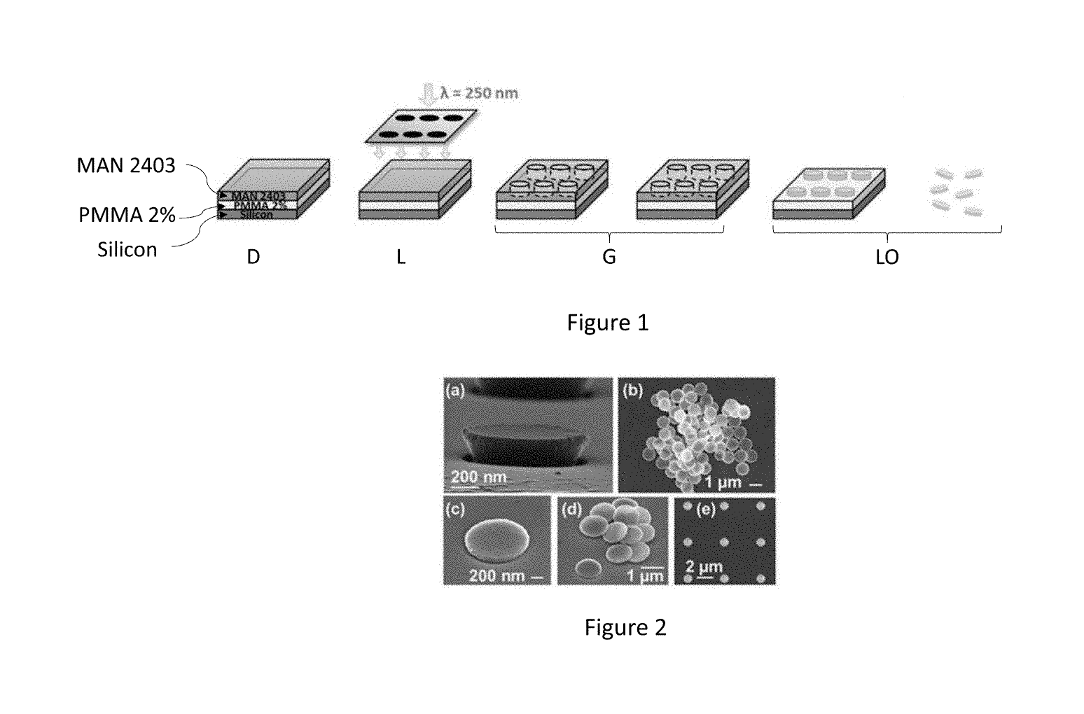

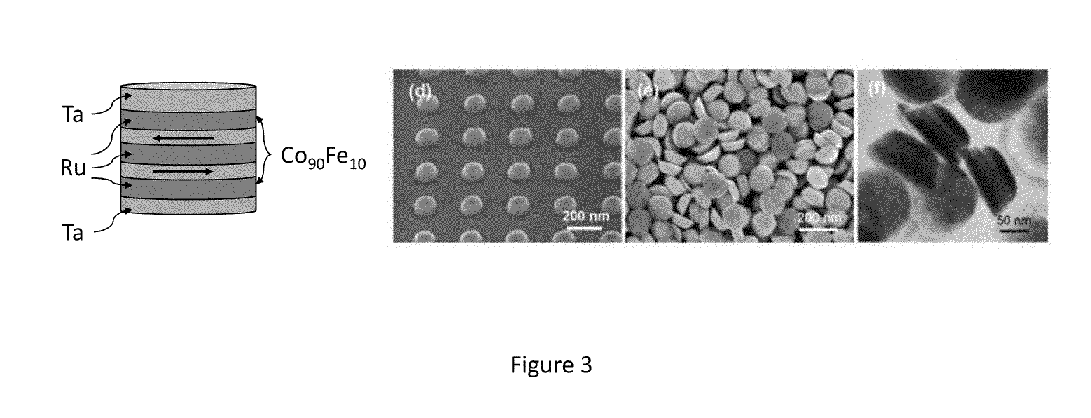

### DETAILED DESCRIPTION

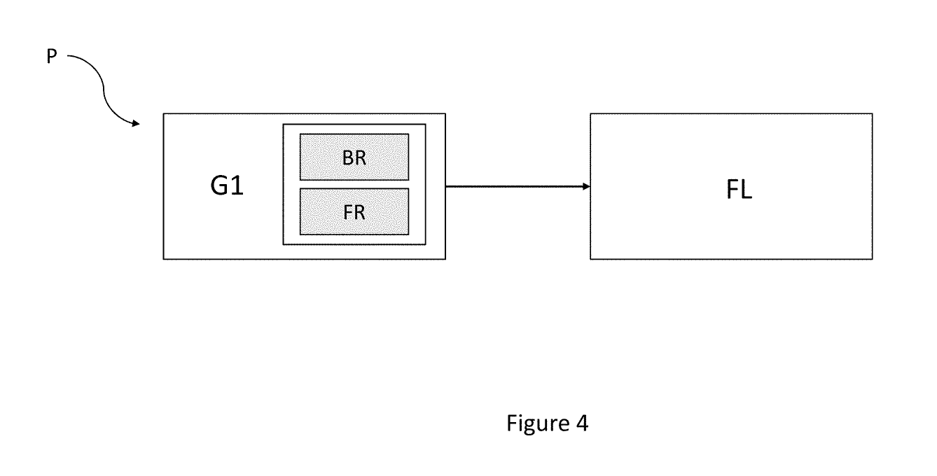

FIG. 4 schematically illustrates the steps of the method for manufacturing a biocompatible fluid including magnetic particles according to an embodiment of the invention.

During step G1, a powder of particles having a final granulometry and intended to be dispersed in a biocompatible fluid is obtained from an initial powder of magnetic particles.

The final granulometry is characterised by a first average size of the particles in a first direction and a second average size of the particles in a second direction.

The initial powder has an initial granulometry characterised by an average size of the particles. The particles of the initial powder may for their part also have an elongated shape.

If the average size of the particles of the initial powder is greater than the characteristic average sizes of the targeted final granulometry, the final powder is obtained by milling BR of the initial powder.

For example, it is possible to mill an initial powder of magnetite particles having an initial average size of 5 μm to obtain a final powder of magnetite particles having an average size of 2 μm.

Alternatively, if the initial powder has an average size of the particles smaller than the average sizes of the target granulometry, it is possible to obtain the final powder by sintering FR of the initial powder.

Beneficially, the steps of milling BR and sintering FR may be carried out in sequence, to adjust the granulometry of the powder until the desired final granulometry is obtained.

The anisotropy of the shapes obtained results both from the size and shape dispersion of the initial powder and from the random character of impacts leading to fracturing of the grains. In the event where the initial particles have an elongated aspect ratio, the particles obtained after moderate milling conserve an elongated aspect ratio (even if this aspect ratio can decrease).

For example, if the powder obtained after the milling step comprises a too small average size of the particles, it is possible to increase the size of the particles by sintering.

Once the final powder has been obtained, it is transferred into a biocompatible fluid during the step FL.

The transfer FL may be carried out by capture of the particles against the wall of a container using a magnet or a magnetic field, emptying the initial liquid and adding a biocompatible fluid.

An alternative consists in recovering the particles by dipping into the container containing them a magnetic device of which the radiation field may be activated or deactivated. The particles will be attracted and maintained against the walls of the device when the radiated field is activated and released into a second biocompatible liquid when the radiated field is deactivated.

According to an embodiment, the method P further includes a step of modification of the granulometry of the particles in solution, after milling or sintering and the transfer of the powder into the biocompatible fluid. This step consists in refining the size distribution of the particles in solution notably by filtration using filters-syringes or paper filters, by suspension/decantation, by magnetic separation, or by centrifugation.

FIG. 5 represents an electron microscope image of the magnetic particles used in the method P according to an embodiment of the invention. The powder has a final granulometry obtained from an initial granulometry by milling/sintering. The particles comprised in the final powder have irregular shapes.

In support of the invention, it is observed that magnetisation measurements on synthetic magnetite crystals, with sizes between 0.3 μm and 30 μm, or on bulk samples of magnetite of natural origin, or on pads lithographed up to diameters of 300 nm indicate a remanence of the magnetisation less than 0.2. See for example, the documents “Grain size dependence of low-temperature remanent magnetization in natural and synthetic magnetite: Experimental study” published in Earth Planet Space 61, 119 (2009) of A. V. Smirnov et al.

These different elements indicate that the magnetic properties of magnetite are suited to the targeted use, that these properties are robust with regard to the shape and the size, and are not very sensitive to the particular elaboration conditions.

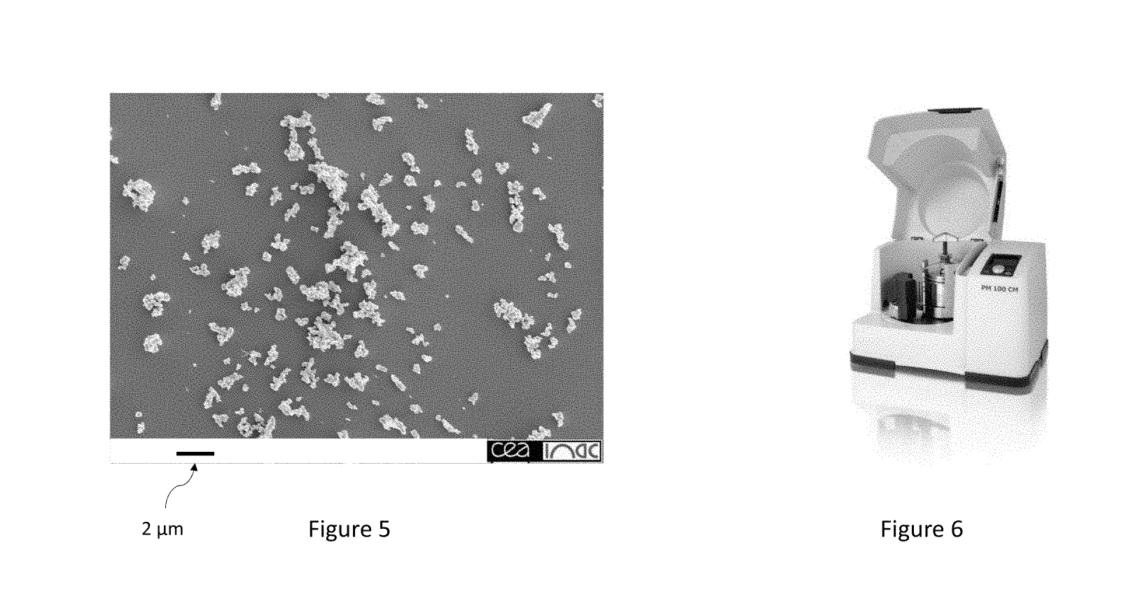

FIG. 6 represents a ball mill, known to those skilled in the art. Such a ball mill is used to modify the initial granulometry by reducing the size of the particles.

Planetary ball mills make it possible, in the case of laboratory models, to manufacture in a single operation, which only lasts several hours, quantities of powder that range from less than one gram (with a single 12 ml vessel) to several hundreds of grams (when several 500 ml vessels are used in parallel). These quantities are several orders of magnitude greater than those obtained by top-down approaches and easily cover the needs for the treatment in parallel of several tumours or tissues, in animals or humans. Furthermore, the quantities are such that it is easily possible to conduct in parallel physical-chemical characterisation studies (e.g. size and surface potential measurements by DLS—Dynamic Light Scattering), or chemical functionalisation studies requiring several milligrams or grams of material.

Other technologies, like the mills used in the pharmaceutical industry, make it possible to mill powder loads of several kilograms up to micronic granulometries. Such volumes fall within the scope of an industrial exploitation of particles, and cannot be envisaged with top-down approaches since the cost price per gram is so high.

The composition of the initial powder is similar to the composition of the desired particles. An alternative consists in carrying out a total or partial oxidation of an iron powder, or to carry out the mechanical synthesis of an iron oxide by milling.

An example of use of a standard ball bill for reducing or modifying the granulometry of a magnetite powder is the following.

The magnetite powder is introduced into a 50 ml milling vessel, made of zirconium oxide, with a certain number of balls of same material, of centimetric diameter. A characteristic of the vessel and the balls is to be constituted of a material of hardness greater than that of the ground powder. For example, the hardness of zirconium oxide on the Mohs scale is 8, that of magnetite is 5.5.

A second characteristic of the vessel and the balls is not to release toxic contaminants during milling. This is the case of zirconium oxide, but it is not the case of steel (which on milling releases chromium). An alternative consists in using a vessel and balls of another hardness, but not releasing toxic contaminants.

The magnetite load represents around one third of the volume of the milling vessel, which represents on average 2 g of magnetite for a 50 ml vessel. A certain quantity of liquid may be added to the load to facilitate the milling thereof, for example 20 ml of isopropanol. Other adjuvants may be added, in variable quantity, for example oleic or stearic acid, which favour the dispersion of the ground particles.

An alternative consists in adding a certain amount of water to induce an oxidation reaction of the particles during milling and to modify the chemical composition thereof.

The vessel is hermetically sealed by a cover. The milling results from the off-centre rotation of the vessel, for example at 600 rpm for 2 hours. The milling times and/or the energy of the balls are adjusted as a function of the initial and final granulom etries.

An alternative consists in using another milling technique or apparatus, different by the type of movement imposed on the milling vessel and by the nature of the impact of the balls with the powder.

At the end of milling and as a function of the conditions used, the granulometry of the powder is reduced, with for example a size distribution such that the largest dimension is centred on 2 μm with a distribution of 30% or more. The shape of the particles is furthermore irregular.

According to an embodiment of the invention, the method P for manufacturing a biocompatible fluid including magnetic particles further comprises a step of chemical functionalisation of the particles.

Very often, the particles are functionalised using compounds that procure a stabilisation of the structure, a protection against oxidation (notably for iron particles), a steric or electrostatic barrier to agglomeration and/or which make it possible to circulate in an organism or a tissue, or to interact with a biological tissue or a cell either to adhere thereto, to penetrate therein, or to deliver therein in a targeted manner a drug or any other active substance.

Beneficially, the grafting of polymers or the encapsulation in silica, used for the stabilisation of the particles, procure a steric repulsion. If, in addition, the functionalised layer or the polymer chains are charged, they induce an electrostatic repulsion between particles, which reduces magnetic attraction effects and increases the dispersion effect.

For example, the steric repulsion effect is obtained by grafting of polyethylene glycol (PEG), which forms a layer of variable thickness on the surface of the particle from 8 nm (PEG 1k) to 15 nm (PEG 5k). The thickness of the PEG layer grafted on the particle imposes a minimum approach distance between the magnetic particles.

If each particle is assimilated with a magnetic dipole, the magnetic interaction energy decreases with the inverse of the cube of the distance d between the particles. The grafting of long molecules on the surface of the particles reduces this interaction effect. The magnetic interaction energy decreasing in 1/d³, this diminishes the effect thereof.

The grafting of PEG also prevents the opsonisation of the particles, which reduces their elimination by phagocytes and extends their lifetime in the organism, see for example the document “Effect of polyethyleneglycol (PEG) chain length on the bio-nano-interactions between PEGylated lipid nanoparticles and biological fluids: from nanostructure to uptake in cancer cells”, published in Nanoscale 6, 2782 (2014) of Pozzi et al.

Beneficially, the functionalisation of the particle may enable the transport of substances for therapeutic use, like the selective targeting of a tissue or a cell, by grafting of antibodies. This functionalisation is ensured by the prior grafting of thiols (for particles covered with gold—most current particles) or amine groups (for the magnetite particles of the invention), see for example the document “Functionalization of Fe₃O₄ NPs by Silanization”, published in Materials. 9, 826 (2016) of S. Villa et al.

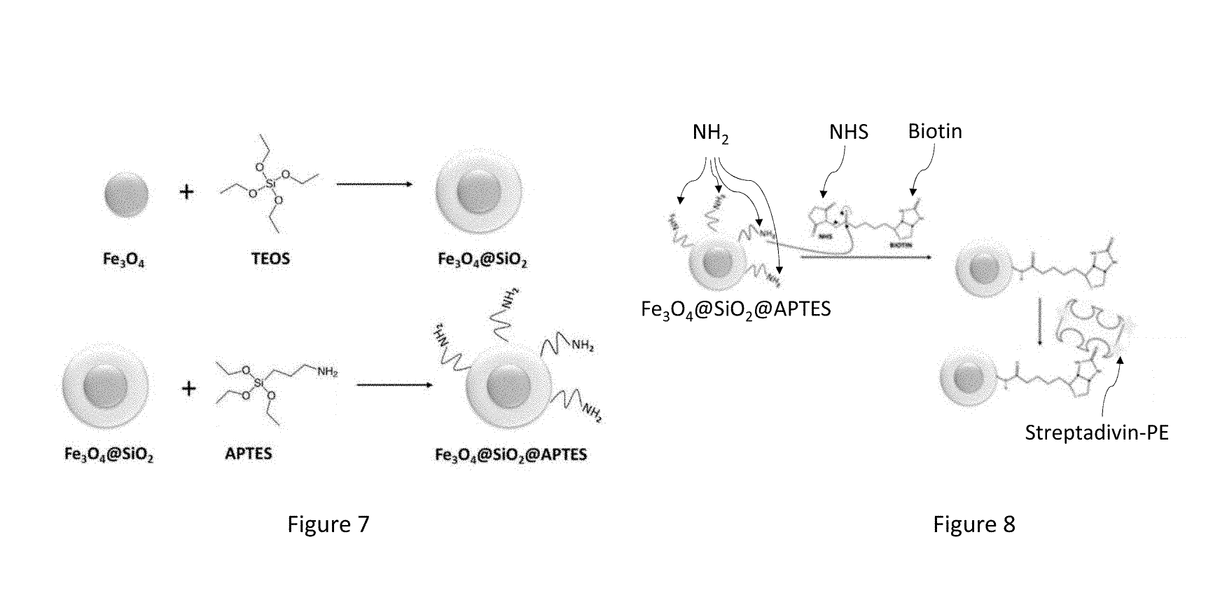

According to a particular embodiment, the functionalisation may be obtained by encapsulation in an inorganic matrix of silica then grafting of an organosilane, as is shown in FIG. 7.

The silica precursor is tetraethoxysilane (TEOS) and the organosilane is 3-aminopropyltriethoxysilane (APTES). The amine functional group of APTES makes it possible to maintain the hydrophilic character of the surface and to graft a biomolecule.

The encapsulation of the particles with silica may be carried out in the following manner: in a two-necked round bottom flask, 6 mg of Fe3O4 particles, 20 ml of absolute ethanol and 100 ml of ultra-pure water, ultrasounds for 15 min at 40° C. Successive addition of 400 μL of ultra-pure water, 900 μL of ammonia solution (28% aq.) and 120 μL of TEOS. Stirring at 40° C. for 2 h.

The functionalisation with APTES is then carried out in the following manner: in a two-neck round-bottom flask, 1 ml of Fe3O4@SiO2 (60 mg/L) in ultra-pure water added to 1 ml of ethanol and 43 μL of APTES (2% v/v). Stirring at 50° C. for 24 h.

The efficiency of the functionalisation of the particles is verified by the grafting of a fluorophore, phycoerythrin (PE) coupled to streptavidin. The cross-linker Nhydroxysuccinimide-Biotin (NHS-Biotin) is used for the formation of an amide bond with the amine group of the APTES, then the streptavidin-PE is bound to the biotin.

For a fluorescence functionalisation, the following method may be used: in a Eppendorf tube the particles are left in contact with 4 μL of NHS-Biotin (10 mM) and 396 μL of phosphate buffered saline at pH 8 (PBS 8), 1 h under vortex. Rinsing three times with PBS 8, twice with PBS 7.4 and suspension in 100 μL of PBS 7.4 then stirring under vortex. Addition of 5 μL of Streptavidin-PE and stirring for 15 minutes under vortex and protected from light. Rinsing three times with PBS 7.4 then deposition on microscope slide for observation with light between 520 and 550 nm.

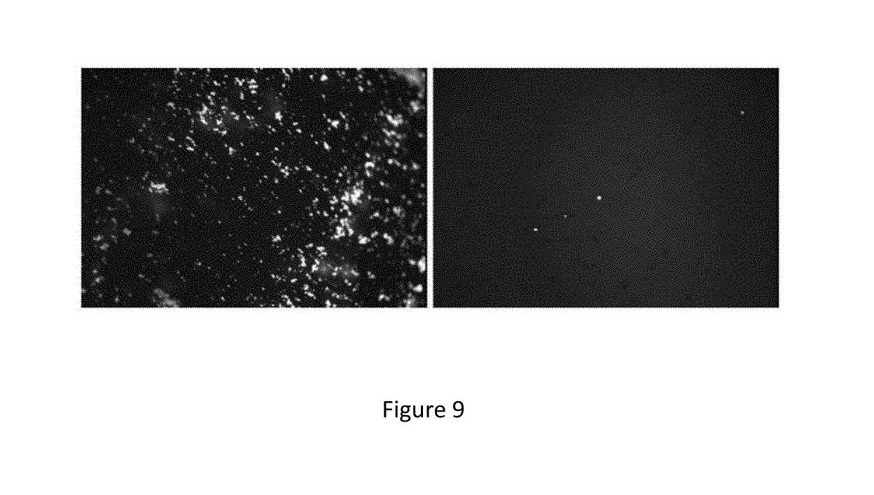

The results of the fluorescence functionalisation are illustrated in FIG. 9, which shows:
On the left, fluorescence optical microscope image of the functionalisation of magnetite particles with APTES;
On the right, fluorescence optical microscope image of non-functionalised control particles.

In these figures a fluorescence emission is observed uniquely for the functionalised particles, which confirms the efficiency of the functionalisation.

The particles intended to be placed in the presence of living tissues or cells are, on coming out of the mill where they are dispersed in isopropanol, conditioned in the following manner. The particles are attracted to the bottom of the container where they are found by means of a magnet; the greatest part of the isopropanol is removed using a pipette: the isopropanol is replaced by ethanol; still while attracting the particles to the bottom of the container and by removing the liquid by pipette, three rinsings using the culture medium are carried out.

The particles are placed in the presence of cells or tissues by direct addition, to the recipient where they are found, of the particle—culture medium solution described. An incubation period, for example 24 hours, may be respected between the placing in presence of the particles and the tissues or the cells to enable the diffusion of the particles within the medium and/or the grafting or the incorporation of the particles on the target species.

The particles intended for microscopic observations, or for measurements or characterisations where it is desirable that they are dispersed on a surface (e.g. magnetic measurements), are, on coming out of the mill where they are dispersed in isopropanol, dispersed in the following manner. The isopropanol is replaced, by the technique described previously, by an inert solvent with high vapour pressure (e.g. acetone). The substrate intended to receive the particles, for example a silica substrate of the order of a square centimetre, is placed in a magnetic field as high as possible, perpendicular to the surface thereof. This may be done by laying the substrate on a powerful permanent magnet. If possible, the substrate is heated to a temperature slightly below the boiling temperature of the solvent. In the case of acetone at ambient pressure, this temperature may be 50° C. A drop of particles in solution is deposited rapidly on the substrate. If the wetting of the drop is rapid and if the evaporation of the drop occurs quickly: 1) the thickness of the drop that spreads/evaporates remains low; 2) the formation of chains of particles, of which the magnetic orientation would here be perpendicular to the surface, is limited by the rapid dispersion on the surface of the substrate and the low thickness of liquid in evaporation. The spreading rate of the drop may also, depending on the nature of the solvent, be accelerated by a surface treatment that increases the substrate/liquid affinity.

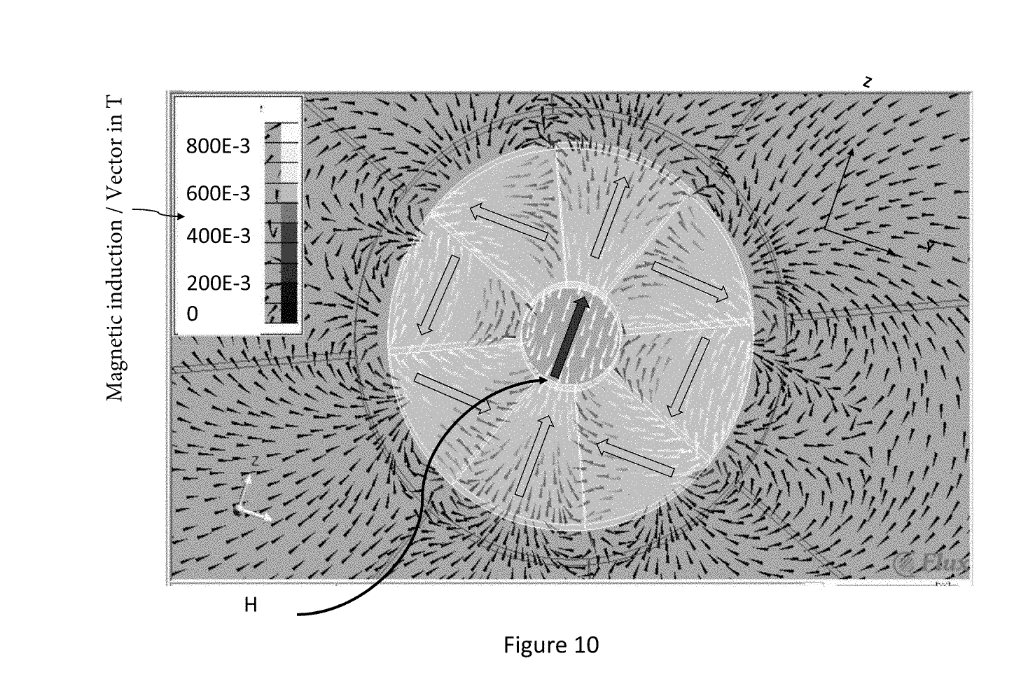

The vibration of the particles (conditioned beforehand and placed in the presence of the tissues or cells to treat according to the described method) is obtained by subjecting them to a field variable in modulus and/or in direction. One solution is to use a Halbach cylinder illustrated in FIG. 10. This cylinder is composed of permanent magnets arranged in sectors and comprises at its centre a cylindrical cavity where a homogeneous magnetic field H reigns, of the order of several tens of teslas, oriented perpendicularly to the axis of the cylinder. The sample of biological tissues or cells to treat, with the magnetic particles, is placed at the centre of the cylinder using the appropriate support, according to whether they are culture cells, living tissues, or potentially a mouse.

The rotation of the cylinder generates a turning field which makes the particles placed in the cavity oscillate, and will lead to a magnetic-mechanical torque.

An aspect of the invention also relates to a biocompatible fluid comprising a powder of magnetic particles of elongated shape. The elongated shape of the particles determines a magnetic shape anisotropy which enables them to be vibrated thanks to the application of a magnetic field variable over time.

The powder of magnetic particles has a final granulometry defined by a first average size of the particles in a first direction and a second average size of the particles in a second direction. The final granulometry is further defined by a first distribution width of the first sizes and a second distribution width of the second sizes. The first average size of the particles is comprised between 0.2 μm and 5 μm. The distribution width of the first sizes is greater than or equal to 30% of the value of the first average size.

Beneficially, the magnetic shape anisotropy enables the efficient vibration of the particles in the presence of a magnetic field variable over time.

According to an embodiment, the second average size is less than 1.5 times the first average size.

Beneficially, this difference between the first average size and the second average size makes it possible to obtain a high magnetic shape anisotropy and thus to increase the magnetic-mechanical torque in the presence of an external variable magnetic field.

According to an embodiment, the magnetic particles are made of iron oxide.

According to an embodiment, ferromagnetic iron oxide is selected from a group including: magnetite, maghemite or a combination of these materials.

Beneficially, these materials are biocompatible and suited to destruction of cancerous human or animal cell or tissue type applications.

The magnetic particles present in the biocompatible fluid may further be chemically functionalised, as explained with reference to the functionalisation step of the method according to an embodiment of the invention.

The biocompatible fluid according to the invention may be used for the destruction of cancerous cells by magnetic-mechanical vibrations according to the following experimental process.

The magnetic particles are transferred into a biocompatible liquid, with a typical concentration of 10⁷ particles/ml.

The particles may be functionalised.

The functionalisation may consist in the grafting of a compound enabling the targeted fastening of the particle on a tissue, a cell or a preferential site of the cell wall.

The grafted compound may be an antibody, which enables the particle to attach itself to the surface of certain specific cells.

The functionalisation may consist in the grafting of a compound that ensures or favours endocytosis of the particles, by the targeted cells.

The functionalisation may have the aim of ensuring better circulation and longer lifetime of the particle in the organism or the tissue, or on the contrary to have as aim to reduce the mobility and ensure the maintaining of the particle as close as possible to the location where it has been positioned, for example during injection within a tissue.

The localisation of the particle at the spot where the magnetic-mechanical vibration has to be exerted is achieved by one or more of the following means: targeted functionalisation; displacement of the particles under the effect of a magnetic field gradient, whether it is internal or external; direct injection within the tissues to treat.

When the particles are in place, they are made to vibrate by the application of a variable external field. An exemplary embodiment is the use of a rotating Halbach cylinder. The Halbach cylinder generates in a cylindrical cavity a magnetic field, perpendicular to the axis of the cavity. The rotation of the cylinder creates a turning field.

The intensity of the magnetic field is of the order of 0.2 T to 1 T. The frequency of rotation of the magnetic field is of the order of 10 Hz to 30 Hz. The duration of a treatment is of the order of 5 minutes to 1 hour.

The treatment may be carried out on culture cells. These cells are for example derived from cancerous cell lines, human or animal. The cells are placed in the presence of the particles, before carrying out the treatment, for an incubation time of a typical duration of 24 hours.

The cells and the particles may be placed in wells, in a suitable nutrient liquid.

The cells and the particles may be integrated in a gel, or in a structure procuring for them a three-dimensional growth substrate.

The aim of the application of the magnetic-mechanical vibrations is to trigger cellular death under application of magnetic-mechanical vibrations inside the cell, on the surface of the cell, or in the medium surrounding the cell. The cells particularly targeted by this application are cancerous cells.

An alternative of the application of the magnetic-mechanical vibrations may be to modify or to orientate cellular division, or to modify or to orient tissue growth. This application aims to promote the regeneration of tissues by the stimulation of their growth, notably those of the spinal cord.

1. A method for manufacturing a biocompatible fluid comprising a powder of magnetic particles of elongated shape having a magnetic shape anisotropy and having a final granulometry, said final granulometry being defined by a first average size of the particles in a first direction and a second average size in a second direction different from the first direction, the second average size being less than 1.5 times the first average size, said final granulometry further being defined by a first distribution width of the first sizes and a second distribution width of the second sizes, said method comprising:
from a powder of magnetic particles having an initial granulometry different from the final granulometry, modifying the initial granulometry by milling and/or by sintering of the powder until the final granulometry is obtained;
introducing the powder of magnetic particles into a biocompatible fluid, the first average size of the magnetic particles being comprised between 0.2 μm and 10 μm and the distribution width of the first sizes representing at least 30% of the value of the first average size.

2. The method for manufacturing a biocompatible fluid according to claim 1, wherein the first average size of the particles is comprised between 0.2 μm and 5 μm.

3. The method for manufacturing a biocompatible fluid according to claim 1, wherein during the modifying of the initial granulometry, the milling of the powder of magnetic particles having the initial granulometry is followed by the sintering of the powder resulting from the milling or the sintering of the powder of magnetic particles having the initial granulometry is followed by the milling of the powder resulting from the sintering.

4. The method for manufacturing a biocompatible fluid according to claim 1, wherein the powder of final granulometry is of same chemical nature as the powder of initial granulometry.

5. The method for manufacturing a biocompatible fluid according to claim 1, further comprising performing a chemical functionalisation of the particles.

6. The method for manufacturing a biocompatible fluid according to claim 5, wherein the chemical functionalisation comprises an encapsulation of at least one part of the particles in an inorganic layer.

7. The method for manufacturing a biocompatible fluid according to claim 6, wherein the inorganic layer is made of silica.

8. The method for manufacturing a biocompatible fluid according to claim 5, wherein the chemical functionalisation includes grafting polymers on the surface of the particles or of the inorganic layer.

9. The method for manufacturing a biocompatible fluid according to claim 8, wherein the grafted polymer includes polyethylene glycol (PEG).

10. The method for manufacturing a biocompatible fluid according to claim 1, wherein the magnetic particles are grains including a metal oxide.

11. The method for manufacturing a biocom patible fluid according to claim 1, further comprising refining the size distribution of the particles in solution.

12. A biocompatible fluid comprising a powder of magnetic particles of elongated shape having a magnetic shape anisotropy and having a final granulometry, said final granulometry being defined by a first average size of the particles in a first direction and a second average size in a second direction different from the first direction, the second average size being less than 1.5 times the first average size, said final granulometry further being defined by a first distribution width of the first sizes and a second distribution width of the second sizes, the first average size of the magnetic particles being comprised between 0.2 μm and 10 μm and the distribution width of the first sizes representing at least 30% of the value of the first average size.
